(19) United States
(12) Patent Application Publication
(10) Pub. No.: US 2018/0056001 A1
(43) Pub. Date: Mar. 1, 2018

(54) SINGLE-USE INJECTOR HAVING A RELEASE ASSEMBLY UNIT FOR SIMPLIFIED ASSEMBLY

(71) Applicant: LTS LOHMANN THERAPIE-SYSTEME AG, ANDERNACH (DE)

(72) Inventors: SARA FORGHANI, URBAR (DE); KARSTEN HEUSER, BAD BREISIG (DE); SEBASTIAN SCHERR, NEUHAEUSEL (DE); UWE WORTMANN, MARBURG (DE)

(73) Assignee: LTS LOHMANN THERAPIE-SYSTEME AG, ANDERNACH (DE)

(21) Appl. No.: 15/793,479

(22) Filed: Oct. 25, 2017

Related U.S. Application Data
(63) Continuation-in-part of application No. PCT/EP2015/059683, filed on May 4, 2015.

(51) Int. Cl. A61M 5/20

(52) U.S. Cl. CPC A61M 5/2033; A61M 5/2455; A61M 2207/10; A61M 2005/208

(57) ABSTRACT

A single-use injector having a plunger actuator which is mounted in a housing, which is loaded by a spring energy store, and which can be unlocked by a movable release device. The plunger actuator can be supported by a traction rod mounted in the housing. The movable release device includes a casing housing. The casing housing includes an upper shell and a lower shell, the separation line of which is oriented in the longitudinal direction of the single-use injector. The casing housing has a cornerless cross-section deviating from a circular shape. The housing is anti-rotationally centered in the casing housing.

### CROSS-REFERENCE TO RELATED APPLICATION

This is a continuation-in-part application of pending international application PCT/EP2015/059683 filed May 4, 2015. The said international application PCT/EP2015/059683 is incorporated herein by reference in its entirety as though fully set forth.

### BACKGROUND OF THE INVENTION

The invention relates to a disposable injector having a piston actuation ram which is supported in a housing and which is loaded by means of a resilient energy store and which can be unlocked by means of a displaceable triggering device, wherein the piston actuation ram can be supported by means of a tensile rod which is supported in the housing.

DE 10 2008 063 519 A1 discloses such a disposable injector. The production and assembly of this disposable injector are complex.

The problem addressed by the present invention is to simplify the production, the assembly and the handling of the disposable injector.

### SUMMARY OF THE INVENTION

This problem is solved with the features of the claims. To this end, the triggering device comprises a covering housing. The covering housing comprises an upper shell and a lower shell whose separation joint is orientated in the longitudinal direction of the disposable injector. The covering housing has a cornerless cross-section which differs from the circular shape. Furthermore, the housing is centered in a rotationally secure manner in the covering housing.

Other details of the invention will be appreciated from the claims and the following descriptions of schematically illustrated embodiments.

### BRIEF DESCRIPTION OF THE DRAWINGS

FIG. 1: is an exploded drawing of a disposable injector;

FIG. 2: is a dimetric illustration of the housing;

FIG. 3: shows a piston actuation ram;

FIG. 4: shows a tensile rod;

FIG. 5: shows a triggering ring;

FIG. 6: shows a support disk;

FIG. 7: shows a preassembly group without any cylinder/piston unit;

FIG. 8: is a longitudinal section of the preassembly group from FIG. 7;

FIG. 9: shows a preassembly group and covering housing prior to the final assembly;

FIG. 10: shows a disposable injector after assembly;

FIG. 11: is a cross-section of the disposable injector from FIG. 10;

FIG. 12: is a longitudinal section of the disposable injector from FIG. 10;

FIG. 13: is a longitudinal section which is normal relative to FIG. 12;

FIG. 14: shows a disposable injector after the triggering;

FIG. 15: shows a disposable injector with an emptied cylinder/piston unit.

### DETAILED DESCRIPTION OF THE INVENTION

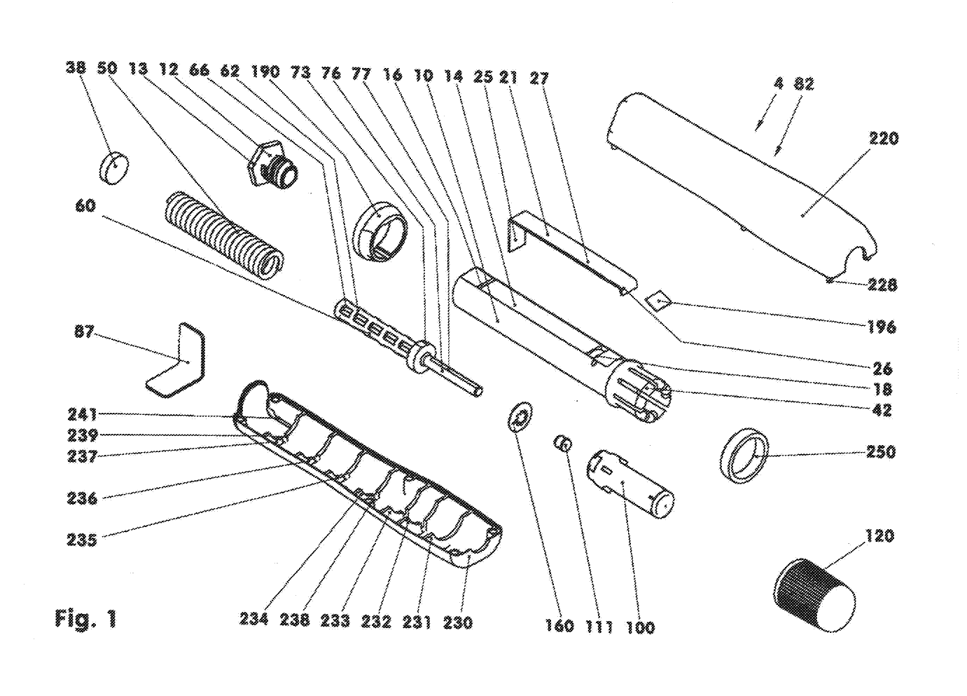

FIGS. 1-15 show a disposable or single-use injector (4). Such injectors (4) are used for the one-off introduction of an injection solution (1) or a solvent into the skin of a patient.

The single-use injector (4) illustrated in FIG. 1 as an exploded view comprises an inner housing (10), a resilient energy store (50), a piston actuation ram (60), a tensile rod (21), a triggering ring (190), a cylinder/piston unit (100) and a covering housing (82).

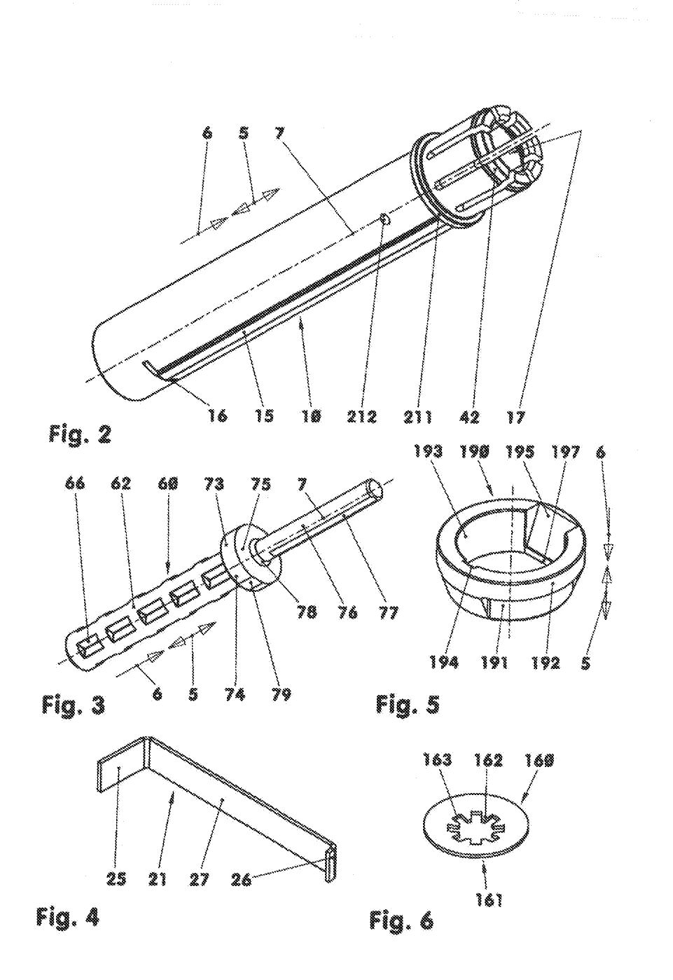

The housing (10), cf. also FIG. 2, has, for example, a length of 127 millimeters. It is tubular and has a substantially cylindrical inner contour. The inner diameter is in the embodiment 15.5 millimeters. In the rear region facing away from the injection location, a single-flight inner thread (11) is arranged, cf. FIG. 8. The outer contour of the housing (10) has a cylindrical basic shape with a diameter of, for example, 20.5 millimeters and with a flattened portion (14). In the embodiment, the flattened portion (14) is flush with the rear end of the housing (10) and has a length of 100 millimeters. In both end regions of the flattened portion (14), the housing (10) has slot-like apertures (16) and (18), wherein the upper aperture (16) extends through the housing (10). With respect to the flattened portion (14), the housing (10) has a guiding rib (15) which is orientated in the longitudinal direction (5). In the front region facing the injection location, the housing has inwardly protruding resilient hooks (42). Between the front and the rear region, an annular housing expansion (211) is arranged. Close to this housing expansion (211) there are arranged in the rear region of the housing (10) assembly openings (212) which extend through the cover of the housing (10).

The resilient energy store (50) is in the embodiment a cylindrical helical compression spring (50) with an outer diameter of, for example, 15.2 millimeters. It is produced from austenitic steel and has along the length thereof a constant wire diameter. The diameter is 2.7 millimeters in the embodiment. The compressed spring (50) has a length of 65 millimeters. In the relaxed state, the helical compression spring (50) is, for example, more than 35% longer than this value.

In FIG. 3, the piston actuation ram (60) is illustrated as a single component. It comprises a, for example, cylindrical guiding journal (62), a ram plate (73) and a piston sliding member (76). The piston actuation ram (60) is produced, for example, from a glass-fiber-reinforced plastics material. The thermoplastic plastics material used is, for example, a polyamide.

The guiding journal (62) is shorter than the length of the compressed helical compression spring (50). It has circle-segment-like recesses (66) which are orientated in a radial direction. These are, for example, distributed over the periphery of the guiding journal (62) and along the length thereof. In the embodiment, the mass of the guiding journal (62) is thereby 10% lower than the mass of a cylindrical guiding journal without recesses. The guiding journal (62) may also be constructed in a tubular manner.

The ram plate (73) of the piston actuation ram (60) is constructed in a disk-like manner and orientated normally with respect to the longitudinal center axis (7) of the disposable injector (4). The diameter thereof is, for example, 14.8 millimeters. It has a conical collar face (75) which faces away from the guiding journal (62). The apex angle of the notional cone of the collar face (75) is, for example, 160 degrees. At mutually opposing sides of the peripheral face (74), the ram plate (73) has a flattened portion (79) in each case.

The piston sliding member (76) is constructed in a rod-like manner and has in the embodiment a diameter of 5.2 millimeters. Two mutually opposing key flats (77) which are orientated in the longitudinal direction (5) each have a length of, for example, 32 millimeters. The collar face (75) of the ram plate (73) merges in a peripheral groove (78) into the piston sliding member (76).

FIG. 4 shows the tensile rod (21), which is produced from a sheet metal strip, for example, with a constant rectangular cross-sectional surface-area. The material thereof is, for example, a rust-resistant and acid resistant steel, for example, X10CrNi18-8 with the material number 1.4310. This austenitic material is for example, used to produce springs. The elasticity modulus thereof is, for example, greater than 190,000 Newton per square millimeter. The length of the tensile rod (21) is in the embodiment 77 millimeters, the width thereof is 10 millimeters and the thickness thereof is 1.2 millimeters. It is constructed in a U-shaped manner and comprises a main member (27), a clamping member (25) and a wrap-around hook (26). The clamping member (25) which is bent, for example, by means of a bending shaping method defines an angle of 90 degrees with the main member (27). The wrap-around hook (26) defines with the main member (27) an angle of 100 degrees. The length of the wrap-around hook (26) is, for example, 20% of the length of the clamping member (25).

The triggering ring (190) which is illustrated in a dimetric view in FIG. 5 is in the embodiment produced from a glass-fiber-reinforced thermoplastic plastics material. This is, for example, polyamide PA 6.6 with 30% glass fiber proportion. The covering face of the triggering ring (190) has an upper cylindrical portion (192) and a lower portion (191). The lower portion (191) is constructed in a partially cylindrical and partially frustoconical manner. The inner wall (193) which is cylindrical in terms of the basic shape has at one side a rotation prevention groove (194) which is orientated in the longitudinal direction (5). At the opposite side, an oblique abutment face (195) is formed. This abutment face (195) is, for example, orientated at an angle of 20 degrees with respect to the longitudinal direction (5). This angle, whose tip in the triggering direction (6) of the disposable injector (4) is located offset with respect to the triggering ring (190), may be between 10 degrees and 45 degrees. The abutment face (195) terminates at a lower shoulder (197). In the region of this lower shoulder (197), the inner wall is delimited by a chord of the basic shape.

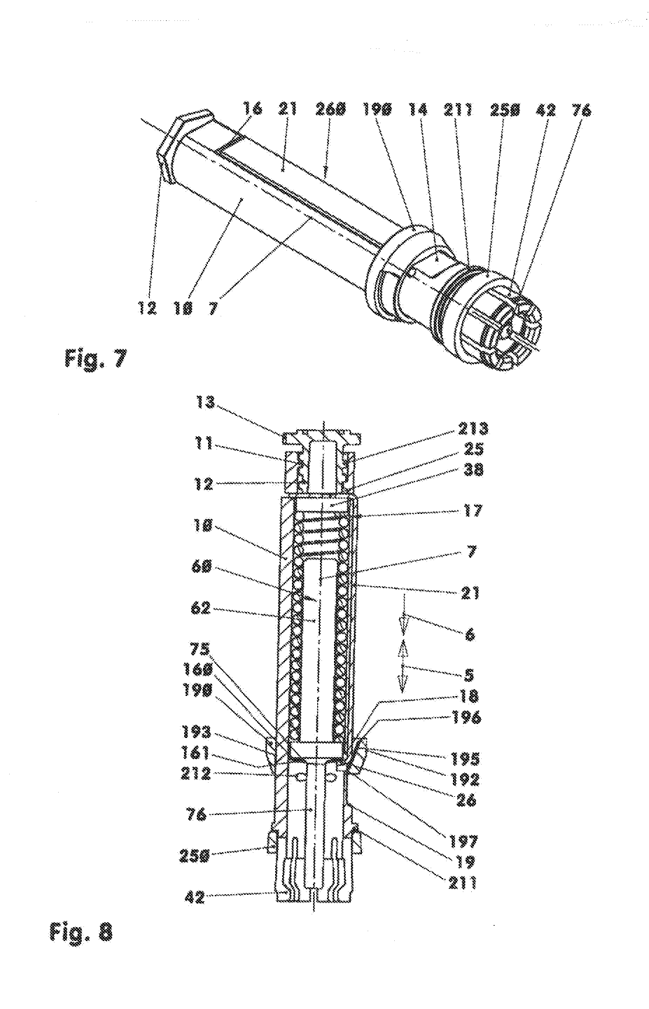

During the assembly, a housing preassembly unit (260) is assembled, cf. FIGS. 7 and 8. In this instance, for example, a support disk (160) is first pushed onto the piston sliding member (76) of the piston actuation ram (60).

In the embodiment, the support disk (160) illustrated as a single component in FIG. 6 is a perforated disk. It is produced, for example, from the same material as the tensile rod (21). Around the central hole there are arranged grooves (163) and wedges (162) by means of which the support disk (160) after the fitting is located in a positive-locking manner on the piston sliding member (76) of the piston actuation ram (60). In the side view, the support disk (160) is constructed in a frustoconical manner. The apex angle corresponds to the apex angle of the collar face (75), against which the support disk (160) is in abutment after fitting to the piston actuation ram (60).

The helical compression spring (50) is fitted to the guiding journal (62) of the piston actuation ram (60). In this instance, the relaxed helical compression spring (50) protrudes beyond the piston actuation ram (62).

An adjustment disk (38) is inserted in the housing (10). The thickness of this cylindrical disk (38) determines the pretensioning of the single-use injector (4). The thicker the adjustment disk (38) is, the higher is the injection pressure of the single-use injector (4). Where applicable, the housing preassembly unit (260) may also be constructed without an adjustment disk (38).

The clamping member (25) of the tensile rod (21) is introduced in the upper housing slot (16) so that the main member (27) faces in the direction of the housing expansion (19). Where applicable, the main member (27) may be in abutment with the flattened portion (14). The tensile rod (21) is consequently arranged in an asymmetrical manner in the housing (10).

The piston actuation ram (60) with the helical compression spring (50) and the support ring (60) is introduced from the side of the resilient hooks (42) into the inner space (17) of the housing (10). The helical compression spring (50) is then in abutment with the adjustment disk (38) and this disk contacts the clamping member (25).

The triggering ring (190) is before or after the piston actuation ram (60) has been introduced—fitted from the rear onto the housing (10). In this instance, a sliding plate (196) is placed in the triggering ring (190). This rectangular sliding plate (196) is, for example, produced from the same material as the tensile rod (21) and has, for example, a thickness of 0.5 millimeters. It is in abutment with the abutment face (195) of the triggering ring (190) and is supported on the shoulder (197). It may optionally be fixed at that location.

When the triggering ring (190) is pushed on, it is guided on the tensile rod (21) and on the guiding rib (15) of the housing (10). The triggering ring (190) is pushed on until it is in abutment with the housing expansion (19) below the rectangular housing recess (18).

A support screw (12) is screwed into the inner thread (11) of the housing (10) until it is in abutment with the clamping member (25) or presses against it. The support screw (12) has, for example, a hexagonal portion (13) for positioning a tool. Where applicable, the thread (213) of the support screw (12) and/or the housing (10) may have a planar tooth arrangement in order to prevent unintentional release of the support screw (12).

The piston actuation ram (60) is now pressed, for example, by means of a tool into the housing (10). The helical compression spring (50) is tensioned. In this instance, for example, the housing (10) is retained on a retention ring (211). The wrap-around hook (26) of the tensile rod (21) is introduced into the rectangular recess (18) and placed at the lower side (161) of the support disk (160) which armors the collar face (75). The triggering ring (190) is pulled upward until it is in abutment with the tensile rod (21). The tensile rod (21) is now supported on the sliding plate (196) which forms an armor-plating of the triggering ring and acts as a locking rod (21). In order to secure the assembly position, it is possible to introduce a, for example, U-shaped curved member into assembly openings (212) of the housing (10). This curved member secures the position of the triggering ring (190) after the clamping device of the resilient energy store (50) has been removed. Even with relatively long-term storage, the resiliently loaded tensile rod (21) does not cause any deformations of the sliding plate (196). Consequently, even after a relatively long period of storage, reliable triggering is ensured.

In the lower region of the housing (10), a securing ring (250) may be placed on the resilient hooks (42) until it is, for example, in abutment with the retention ring (211). In this instance, the splayed resilient hooks (42) are pressed together so that, after the assembly, the securing ring (250) is located with a clearance fit in a non-releasable manner on the housing (10). This housing preassembly unit (260) may now, for example, be conveyed to another workplace.

In another production line, the cylinder/piston unit (100) is, for example, produced and filled. The cylinder/piston unit (100) comprises a, for example, transparent cylinder (101) and a piston (111) which is guided in the cylinder (101). In the upper region, the cylinder (101) has a peripheral collar (108). The discharge opening (106) of the cylinder/piston unit (100) arranged at the end face (103) is constructed as a short, cylindrical, nozzle-like hole (106). The diameter of this hole (106) is, for example, 0.18 millimeters.

After the cylinder (101) has been filled, the piston (111) which is then located with a press-fit in the cylinder (101) is inserted. The closure cap (120) can now be positioned. This has on the peripheral face (122) thereof a fluting (123) in order to prevent fingers from sliding off. The piston/cylinder unit (100) which has been provided in this manner can now be stored until further assembly.

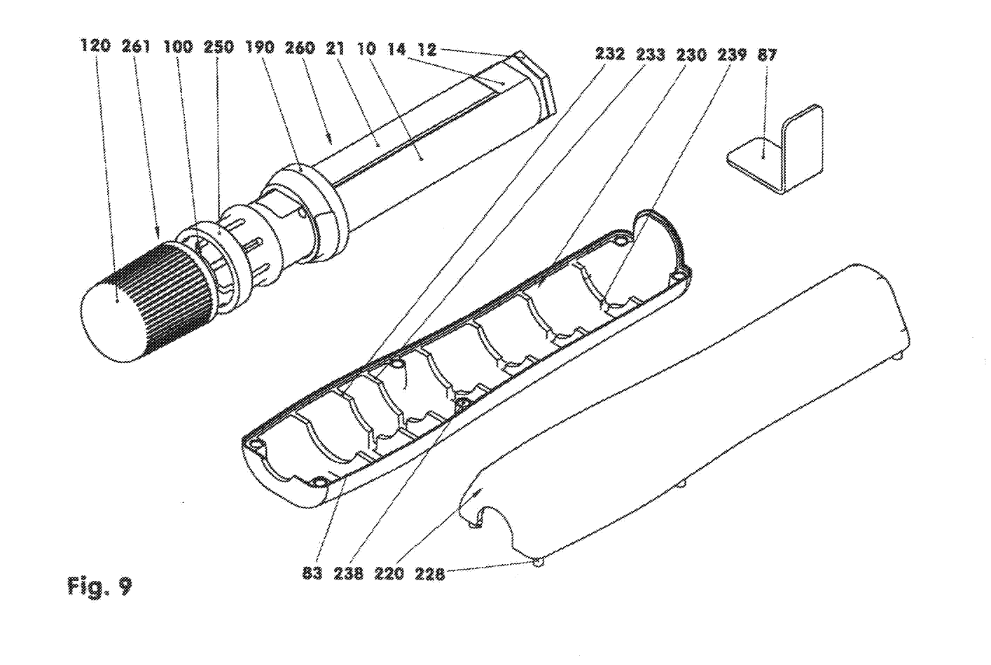

Prior to the final assembly, the cylinder/piston unit (100) is inserted into the housing preassembly group (260), cf. FIG. 9. In this instance, the cylinder/piston unit (100), with the resilient hook (42) being expanded, is inserted into the housing (10) until the resilient hooks (42) engage behind and lock the collar (108). The resilient hooks (42) act as catch hooks (42). Now, the securing ring (250) is pushed forward and presses the catch hooks (42) against the cylinder/piston unit (100). The cylinder/piston unit (100) is now retained in the housing (10) in a non-releasable manner. The closure cap (120) is spaced apart from the housing (10). This subassembly (261) can be handled, transported and stored.

The covering housing (82) comprises in the embodiment an upper shell (220) and a lower shell (230). The separation joint (83) of both shells (220, 230) is orientated in the longitudinal direction (5) of the disposable injector (4).

The two shells (220, 230) of the covering housing (82) have at the inner side thereof in each case reinforcement ribs (221-227; 231-237). These transverse ribs (221-227; 231-237) are orientated normally with respect to the longitudinal direction (5) of the single-use injector (4). In this instance, the rear reinforcement ribs (223-227; 232-237) each have two portions between which a groove (239) is arranged. The foremost reinforcement ribs (221, 222; 231) facing away from the operator are each constructed in an interruption-free manner. The upper shell (220) and the lower shell (230) are connected to each other, for example, by means of a plurality of journal connections (228, 238). In this instance, in the embodiment, the upper shell (220) has at the separation joint (83) six journals (228) which engage in journal holes (238) of the lower shell (230). Where applicable, the journal connections (228, 238) may engage with each other during the assembly. Adhesive bonding of the lower shell (230) to the upper shell (220) is also conceivable.

The housing (10) and the covering housing (82) are, for example, produced from plastics material. This may be a thermoplastic or thermosetting material, for example, POM, ABS, etcetera.

During the final assembly of the disposable injector (4), the housing preassembly group (260) is placed with the cylinder/piston unit (100), for example, in the lower shell (230). In this instance, the rib (15) of the housing (10) is longitudinal groove (239) of the lower shell (230). The triggering ring (190) is located between the second transverse ribs (222, 232) and the third transverse ribs (223, 233). The head of the support screw (12) protrudes beyond the rearmost transverse rib (227; 237). The closure cap (120) is located outside the covering housing (82). There is inserted into the slot (241) the securing element (87) which, for example, is clamped between the securing screw (12) and the covering housing (82). The support screw (12) can be secured against further rotation, for example, in a positive-locking manner. Where applicable, an additional compression spring between the support screw (12) and the covering housing (82) can increase the resistance against unintentional triggering. This spring also determines the resistance of the disposable injector (4) during triggering. The U-shaped curved member can be removed.

In order to complete the assembly, the upper shell (220) is placed on the lower shell (230) and secured, for example, by means of adhesive bonding, engagement, etcetera. Now, an additional originality closure, not shown in the drawings, for example, a banderole may be fitted over the covering housing (82) and the closure cap (120). It is also conceivable for the assembly to be carried out in a different sequence from the one described.

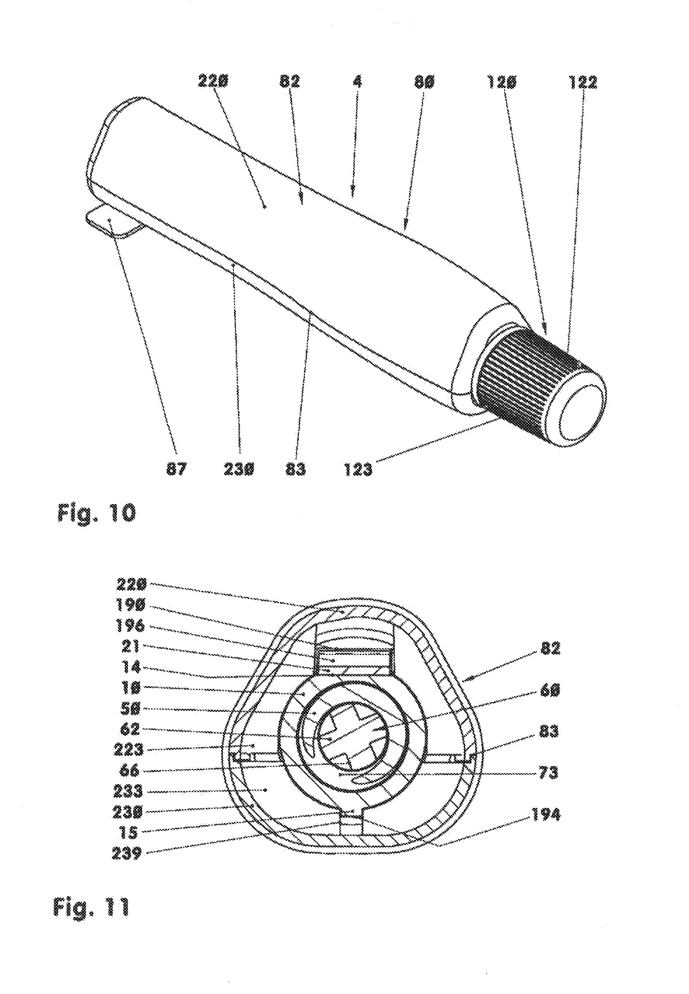

The completely assembled disposable injector (4) is illustrated in FIG. 10 as a dimetric view and in FIG. 11 in cross-section. The cross-section of the covering housing (82) has no corners and edges and deviates from a circular shape. In the embodiment, the cross-section is constructed in a similar manner to a regular triangle with rounded corners. However, it is also conceivable to construct the cross-section in a manner similar to a polygon.

The cross-sectional surface-area in the rear region of the disposable injector (4) is 70% of the cross-sectional surface-area in the front region of the disposable injector (4) facing the injection location. The constant increase of the cross-sectional surface area is located, when viewed from the rear, in the third quarter of the length of the disposable injector (4). Consequently, the disposable injector (4) can be readily gripped by the operator. For example, he encloses the cover housing (82) with all his fingers. In this instance, the outlet opening (106) may be located, for example, at the side of the index finger or at the side of the little finger.

The fully assembled disposable injector (4) may now be packaged and marketed. If, for example, it is placed on a table after unpacking, as a result of the housing geometry there is no risk of it rolling away.

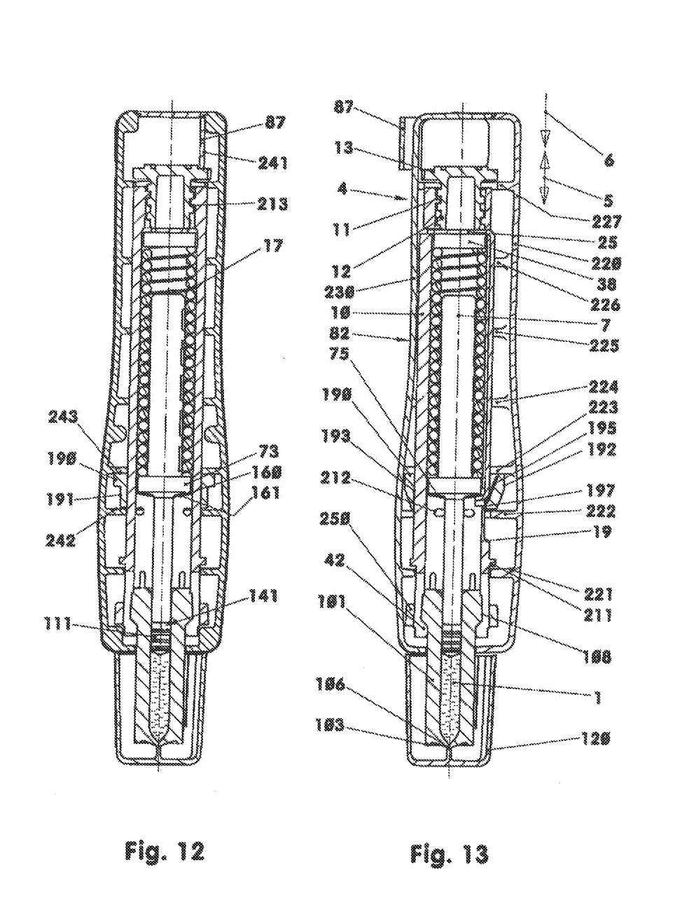

The disposable injector (4) which is illustrated in FIGS. 12 and 13 in longitudinal sections which are orientated normally with respect to each other comprises the covering housing (82) in which the inner housing (10) with the piston actuation ram (60) and the helical compression spring (50) as a permanently loaded resilient energy store (50) and the cylinder/piston unit (100) are arranged. The piston actuation ram (76) is spaced apart from the piston (111) by means of an intermediate space (141). The cylinder/piston unit (100) is closed by means of the protective cap (120). The triggering of the single-use injector (4) is prevented by means of the securing sliding member (87). In this instance, the side of the transverse ribs (222, 231) facing the triggering ring (190) forms a locking flank (242).

Before the single-use injector (4) is used, the originality closure is first removed. After the closure cap (120) has been removed, the securing element (87) can be pulled out. The single-use injector (4) is now ready for use and is, for example, placed on the skin of the patient. In this state, the self-locking between the locking rod (21) and the triggering ring (190) also prevents unintentional self-triggering of the disposable injector (4).

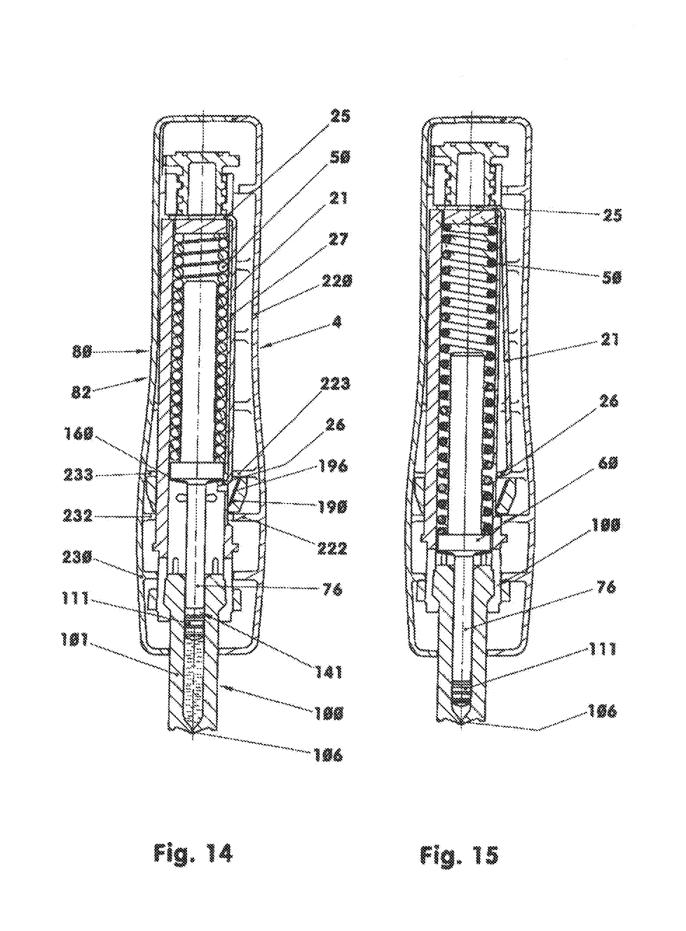

In order to trigger the single-use injector (4), the triggering device (80) is actuated by the operator. The operator presses the covering housing (82), which forms a triggering element in the structural form of a triggering sleeve (82), in the triggering direction (6) relative to the housing (10) in a forward direction, that is to say, in the direction of the skin of the patient. The triggering sleeve (82), in this instance by means of the side of the transverse rib (222, 233) facing the triggering ring (190), which rib forms a triggering flank (243), displaces the triggering ring (190) downward relative to the housing (10), cf. the sectioned illustrations of FIGS. 12-15. In FIG. 14, the non-static state directly after the triggering is illustrated. The resilient energy store (50) presses the ram plate (73) forward. In this instance, the pressure disk (160) displaces the wrap-around hook (26) of the locking rod (21). The locking rod (21) slides along the sliding plate (196) in an outward direction and consequently completely releases the piston actuation ram (60). In this instance, the locking rod (21) may where applicable strike an insulating rubber layer. The piston actuation ram (60), in a state loaded by the relaxing resilient energy store (50), rapidly moves forward or downward. The piston sliding member (76) strikes the piston (111) and pushes it forward. In this instance, the air is displaced from the intermediate space (141) along the key flats (77). The injection solution (1) which is stored in the cylinder (101) is displaced through the outlet opening (106) and the hard skin of the patient into the body of the patient.

FIG. 15 shows the single-use injector (4) after the triggering. The triggering device (80) and the triggering ring (190) are displaced downward relative to the housing (10). The limit stop of the triggering sleeve (82) on the securing ring (250) prevents the triggering device (80) from being pushed further out relative to the housing (10). The locking rod (21) is displaced outward. In this instance, it blocks, for example, the release sleeve (82) with the triggering ring (190) from being pushed in again. The resilient energy store (50) is relaxed. The piston actuation ram (60) is located in the front end position thereof. The cylinder/piston unit (100) is emptied.

Of course, it is also conceivable to combine the different embodiments mentioned with each other.

### LIST OF REFERENCE NUMERALS

1 Injection solution, water for injection purposes
4 Single-use injector, disposable injector
5 Longitudinal direction
6 Triggering direction
7 Longitudinal center axis
10 Housing
11 Inner tread
12 Support screw
13 Hexagonal portion
14 Flattened portion
15 Guiding rib
16 Housing opening, slot-like
17 Inner space
18 Housing opening with rectangular cross-section
19 Housing expansion
21 Support rod, locking rod
25 Clamping member
26 Wrap-around hook
27 Main member
38 Disk, adjustment disk
42 Catch hook, resilient hook
50 Resilient energy store, helical compression spring, spring
60 Piston actuation ram
62 Guiding journal
66 Recesses
73 Ram plate
74 Peripheral face
75 Collar face
76 Piston sliding member
77 Key flats
78 Grooves
79 Flattened portion
80 Triggering device
82 Covering housing, triggering element, triggering sleeve
83 Separation joint
87 Securing sliding member, securing element
100 Cylinder/piston unit
101 Cylinder
103 End face
106 Hole/discharge opening
108 Collar
111 Piston
120 Closure cap
122 Peripheral face
123 Fluting
141 Intermediate space
160 Support disk
161 Lower side
162 Wedges
163 Grooves
190 Triggering ring
191 Lower region
192 Cylindrical region
193 Inner wall
194 Rotation prevention groove
195 Abutment face
196 Metal insertion sheet, sliding plate
197 Shoulder
211 Retention ring
212 Assembly openings, securing element apertures
213 Thread
220 Upper shell
221, 222 Reinforcement ribs, transverse ribs, one-piece
223-227 Reinforcement ribs, transverse ribs, two-piece
228 Journal
230 Lower shell
231 Reinforcement rib, transverse rib, one-piece
232-237 Reinforcement rib, transverse rib, two-piece
238 Journal holes
239 Longitudinal groove
241 Slot
242 Locking flank
243 Triggering flank
250 Securing ring
260 Housing preassembly unit, housing preassembly group
261 Subassembly

What is claimed is:

1. A disposable injector (4) having a piston actuation ram (60) which is supported in an inner housing (10) and which is loaded by a resilient energy store (50) and which can be unlocked by a displaceable triggering device (80), wherein the piston actuation ram (60) can be supported by a tensile rod (21) which is supported in the inner housing (10), characterized in
that the displaceable triggering device (80) comprises a covering housing (82),
in that the covering housing (82) comprises an upper shell (220) and a lower shell (230) having a separation joint (83) is orientated in the longitudinal direction (5) of the disposable injector (4),
in that the covering housing (82) has a cornerless cross-section which differs from a circular shape, and
in that the inner housing (10) is centered in a rotationally secure manner in the covering housing (82).

2. The disposable injector (4) as claimed in claim 1, characterized in that the inner housing (10) comprises a displaceable triggering ring (190) which is arranged exteriorly on the inner housing (10) between a locking flank (242) and a triggering flank (243) of the covering housing (82).

3. The disposable injector (4) as claimed in claim 2, characterized in that there is arranged in the covering housing (82) a preassembly unit (260) which comprises the inner housing (10) with the resilient energy store (50), the piston actuation ram (60), the tensile rod (21) and the displaceable triggering ring (190).

4. The disposable injector (4) as claimed in claim 1, characterized in that the tensile rod (21) is constructed in a u-shaped manner and is arranged in an asymmetrical manner on the inner housing (10).

5. The disposable injector (4) as claimed in claim 4, characterized in that the tensile rod (21) is constructed in a strip-like manner and is supported on the inner housing (10) by a clamping member (25).

6. The disposable injector (4) as claimed in claim 5, characterized in that the inner housing (10) has an inner thread (11) in which a support screw (12) which clamps the tensile rod (21) is screwed.

7. The disposable injector (4) as claimed in claim 1, characterized in that the inner housing (10) has securing element apertures (212).

8. The disposable injector (4) as claimed in claim 1, characterized in that it comprises a cylinder/piston unit (100) which is arranged in a state secured in the inner housing (10) by catch hooks (42) and a securing ring (250) which surrounds them.

9. The disposable injector (4) as claimed in claim 1, characterized in that an adjustment disk (38) is arranged between the resilient energy store (50) and the inner housing (10).

10. The disposable injector (4) as claimed in claim 1, characterized in that the inner housing (10) is supported in the covering housing (82) by an unlockable securing element (87).
